(19) United States
(12) Patent Application Publication
(10) Pub. No.: US 2026/0122456 A1
(43) Pub. Date: Apr. 30, 2026

(54) SYSTEM AND METHOD OF A MONITOR OPERATING AS AN ANCHOR

(71) Applicant: Industrial Scientific Corporation, Pittsburgh, PA (US)

(72) Inventors: Tae Yeon Won, Mars, PA (US); Laura Jean Faust, Pittsburgh, PA (US); Jonathan Eric Dinsmore, Aliquippa, PA (US); Justin Allen Bee, Follansbee, WV (US); Grayham Matthew Grega, Imperial, PA (US); Ajaykumar Raju Bhupathiraju, McDonald, PA (US); Alan Scott Lofstead, Canonsburg, PA (US); Lisa Ann McCracken, Oakdale, PA (US); Joshua Lazzari, Canonsburg, PA (US); Sachin Shah, Troy, MI (US)

(21) Appl. No.: 19/401,841

(22) Filed: Nov. 26, 2025

Related U.S. Application Data
(63) Continuation of application No. PCT/US2025/040966, filed on Aug. 6, 2025.
(60) Provisional application No. 63/712,661, filed on Oct. 28, 2024.

(51) Int. Cl. H04W 4/38; G01N 33/00; H04L 67/12; H04W 40/12; H04W 84/18

(52) U.S. Cl. CPC H04W 4/38; G01N 33/0075; H04W 40/12; H04L 67/12; H04W 84/18

(57) ABSTRACT

A system includes a cooperative monitoring group of gas monitors including a network of end points including at least one end point from each class selected from: a gas monitor class including a gas sensing element and a wireless communication interface, a gas station class including at least one gas monitor physical interface, and a wireless communication interface, and a data manager class including a wireless communication interface and an external communication interface. The cooperative monitoring group is configured to operate as a mesh network, and external communications of the cooperative monitoring group from end points of the gas station class and the gas monitor class are routed through end points of the data manager class. The system includes a fence-line comprising a plurality of gas monitors at selected locations.

### CROSS-REFERENCE TO RELATED APPLICATIONS

This application claims priority to and is a continuation of International Patent Application Serial No. PCT/US2025/040966, filed Aug. 6, 2025 (Attorney Docket No. ISCI-0058-WO).

International Patent Application Serial No. PCT/US2025/040966 claims the benefit of and priority to U.S. application Ser. No. 63/712,661 , filed on Oct. 28, 2024 (Attorney Docket No. ISCI-0056-P01).

Each of the foregoing applications is incorporated herein by reference in its entirety for all purposes.

### BACKGROUND

Gas monitoring at various types of industrial facilities is crucial to maintaining a safe environment, protecting workers, and ensuring proper operation of facilities. Previously known gas monitoring systems suffer from a number of drawbacks. For example, increasing electronic demands on monitoring devices compete with the desire for long battery life, and reduction of time spent on power management of gas monitors. In another example, devices need to be calibrated and maintained, and the workflows to perform these operations are burdensome to the ongoing operation of the facility and for individual operators using a gas monitor. Previously known systems do not provide for convenient and ready monitoring environments that can support arbitrary monitoring, for example in locations such as enclosed spaces, that have high and convenient availability, and that are integrated into the general alarm system for the gas monitoring system. Further, previously known systems are challenged by the flexibility required to support different industrial facilities with different constraints and implementation challenges, where significant challenges are raised in designing systems to meet the needs of some facilities without forcing excessive cost into the solution for features that are not needed.

### SUMMARY

The disclosure herein provides numerous benefits with respect to addressing the challenges of managing gas monitoring of industrial facilities. Forming cooperative monitoring groups including long term independent anchors results in improved coverage of the facility and improved compliance while reducing power needs. Leveraging a gas monitor class which can serve a dual role of gas monitor or anchor provides for ease of managing temporary or transient changes to the facility, and provides for detection paradigms that would otherwise be unavailable or impractical. The dual role has numerous benefits with respect to meeting fence-line challenges, including being able to geofence a region based on power, signal strength, location, sensor set, or any other aspect of a monitor. Utilizing sensor/board ID numbers and manufacturing with dummy code for operability and then downloading the latest information and calibration for the instrument on location has the benefit of improving sensor-specific configuration and managing the history of that sensor which is currently an onerous process. Other benefits may also be present, and embodiments disclosed herein may not have certain or all of the described benefits. A person of skill in the art having the benefit of this disclosure will understand that the disclosed embodiments are especially suited for gas monitoring at various types of industrial facilities with devices and systems having improved power management, improved calibration and maintenance operations, as well as other improvements described herein.

### BRIEF DESCRIPTION OF THE FIGURES

FIG. 1 schematically depicts an example gas monitoring system for an industrial facility.

FIG. 2 schematically depicts an example gas monitoring system for an industrial facility.

FIG. 3 schematically depicts an example gas station in external communication.

FIG. 4 schematically depicts an example gas monitoring system for an industrial facility.

FIG. 5 schematically depicts an example gas monitoring system for an industrial facility.

FIG. 6 schematically depicts an example gas monitoring system for an industrial facility.

FIG. 7 schematically depicts an example gas monitoring system with a monitoring device currently depicted off location.

FIG. 8 depicts a number of example monitoring devices.

FIG. 9 depicts a number of example monitoring devices.

FIG. 10 depicts a gas station component, having two different monitor components docked thereon.

FIG. 11 depicts an example system.

FIG. 12 depicts a gas monitor class.

FIG. 13 depicts a gas station class.

FIG. 14 depicts a data manager class.

FIG. 15 depicts an example gas monitor physical interface of the gas station class.

FIG. 16 depicts an example procedure.

FIG. 17 depicts an example procedure.

FIG. 18 depicts an example procedure.

FIG. 19 depicts a selected group of the plurality of gas monitors.

FIG. 20 depicts an example procedure.

FIG. 21 depicts an example system that includes distribution of instrument calibration operations.

FIG. 22 depicts sensor history values.

FIG. 23 depicts sensing calibration values.

### DETAILED DESCRIPTION

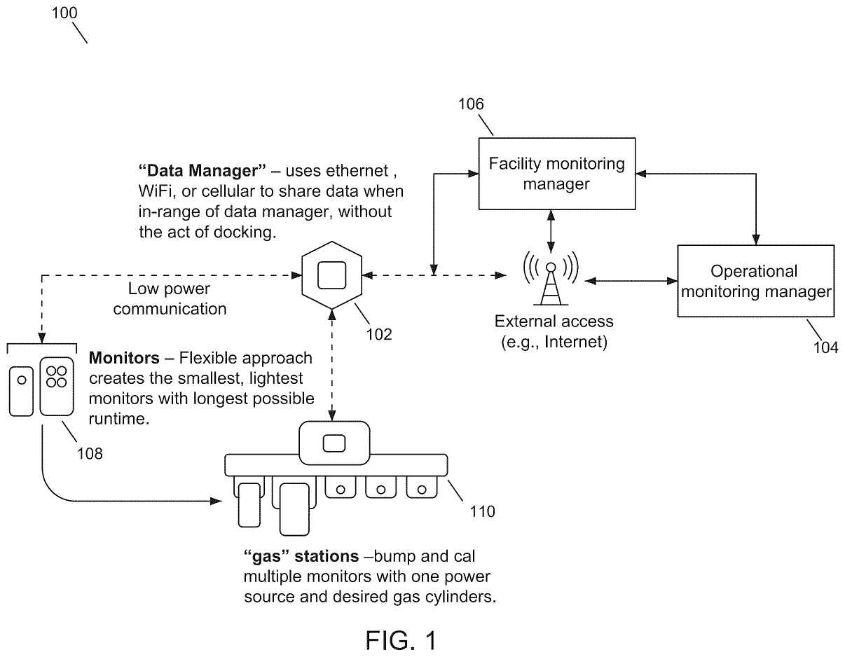

Referencing FIG. 1, an example gas monitoring system 100 for an industrial facility is schematically depicted. The example system utilizes multiple classes of participants on a network supporting the gas monitoring system. The utilization of multiple classes of participants allows for certain participants to be configured to meet the needs of that participant class to support the gas monitoring system, while minimizing the costs and implementation complications introduced by previously known systems, modular systems, or uniquely configured systems.

An example participant class includes at least a data manager class component 102 that manages external communications for the system (e.g., communications to the operational monitoring manager 104 and/or to the facility monitoring manager 106, in the example of FIG. 1). External communications, as utilized herein, include any communications that go to devices apart from the facility, and/or apart from a portion of the facility having the monitored aspects (e.g., where the industrial process that is being monitored is located).

External communications may include communications performed using cellular communications, satellite communications, internet communications, wide area network communications, LAN, cloud, or the like. Various embodiments of the present disclosure include other device classes that may have external communications capability, and/or the data manager class component has additional capabilities in certain embodiments.

Another example participant class includes at least a monitoring class component 108 that monitors at least one value in the physical location of the monitor. In certain embodiments, the monitoring class component includes a gas monitor that is responsive to at least one gas species, but other types of monitors, for example including monitors for temperature, radiation, noise/vibration, radiation, or other aspects of interest in the environment are contemplated herein. In certain embodiments, a typical utilization of the monitoring class component is an operator at the facility that carries the monitor on their person. In certain embodiments, a monitoring class component may be positioned at a location for location based monitoring rather than, or in addition to, personal monitoring.

Another example participant class includes a gas station class component 110 (or a docking station class component). The example gas station class component provides support for the monitoring class component(s), and can manage calibration operations and/or rationality checks (e.g., performing a bump test to ensure monitor operation) for the monitoring class components. In certain embodiment, the gas station class component provides for charging operations and/or charge management for monitoring class components. In certain embodiments, a separate charging class component may be provided in the system, for example allowing gas monitors to be charged separately from the gas station class component, and/or as a part of the gas station class component.

Another example participant class includes an anchor class component. The anchor class component provides for extension of the low power network to reach devices, to continue connectivity through a selected area of the facility, and/or to improve availability of communication support in selected locations, challenging areas, for temporary operations, or the like. In certain embodiments, the class scheme provides for limited power requirements from an anchor class component to allow the anchor component to perform operations for an extended period, for example up to three years, operating off of only battery power. Accordingly, anchor placement at a facility does not require consideration of available infrastructure (e.g., power) or installation of additional infrastructure to support monitoring operations. Accordingly, monitoring support for a facility can be rapidly designed and installed with a high confidence of success. Further, monitoring support for a facility can rapidly change with the facility, and grow with the facility, without the need for a redesign. Further, scaling of monitoring operations can be performed by adding to the system, without having to replace base infrastructure or components that are already in place.

The description herein utilizing component classes for a monitoring facility is non-limiting and provided to illustrate certain aspects of the disclosure. A given facility utilizes at least a data manager class component and a number of monitoring class components. The other classes are optional and non-limiting, and may be included according to the goals of the system, including the monitored facility size, number of operators, distribution of operators and/or monitored areas throughout the monitored facility, economic priorities of the operating facility (including, e.g., operational costs, capital expenditures, labor costs, etc.), and/or the monitoring needs of the facility (e.g., personal detection vs. area detection, parameters monitored). Embodiments herein also provide for rapid configuration of the monitoring system to support changes in the monitoring needs of the facility, including operational changes that may be temporary or long term.

Further, a given component may have additional capabilities beyond the baseline for the class, and a given component may have sufficient capabilities to be considered within more than one class. For example, an anchor class component may have a sensor thereon, and could be considered as either an anchor class component or a monitoring class component. In another example, a monitoring class component may have a cellular connection capability, and could be considered as an anchor class component, a monitoring class component, and/or a data manager class component. In certain embodiments, the class of a specific component may depend upon the specific configuration of the component, the operating condition of the facility, the way the component is being utilized, and/or the specific operations being performed. For example, a monitoring component may be utilized as an anchor, and be treated as an anchor class component during that use. The description herein describing components as in particular classes is provided to illustrate aspects of the present disclosure, but it will be understood that a given component may be considered in one class for a first system and in a second class for a second system, and/or the given component may be considered in one class for a first operation, and in a second class for a second operation.

The example of FIG. 1 utilizes an operational monitoring manager, for example that allows an external user to ensure that all devices (e.g., monitoring class components) are in a known location (and/or it is expected that the location may not be presently known), and/or to gather performance and operational data from the gas monitoring system (e.g., collecting monitored data; compliance data such as calibration, bump, or utilization operations; alarms, pre-alarms, and/or other events; performance data relating to the low power network, etc.). In certain embodiments, the operational monitoring manager is embodied on a cloud server at least intermittently in communication with the gas monitoring system (e.g., through the data manager component), and may be operated by a provider of the gas monitoring system. The example of FIG. 1 utilizes a facility monitoring manager, for example that allows someone related to the facility (e.g., a safety office, compliance officer, facility manager, etc.) to receive just the desired information, potentially through an interface set up by and/or configured by someone utilizing the operational monitoring manager. In certain embodiments, the functions of the facility monitoring manager and/or the operational monitoring manager may be combined into a same unit, and/or may be further divided. The facility and/or operational monitoring manager(s) may be positioned separate from the facility, in a separate location on the facility, and/or otherwise remotely positioned in any selected location that is at least intermittently communicatively coupled to the gas monitoring system.

Example features of a “data manager”, “monitor”, and “gas station” are depicted on FIG. 1. Any such descriptions are a non-limiting example. In the example of FIG. 1, the data manager provides external data communication for the gas station, for example ensuring that firmware on the gas station is up to date, and executing operations for updates as indicated. Further, the data manager performs firmware updates for monitoring devices, and/or ensures that the gas station has up to date firmware available for relevant devices. In certain embodiments, gas monitors provide communications with the data manager over the low power network when the gas monitor is within range of the data manager. For example, confirmation of operation, any alerts, alarms, or anomalous readings that the gas monitor has encountered since the last data synch with the data manager, or the like. In certain embodiments, the data manager directly checks the firmware and/or calibration status of the gas monitors. In certain embodiments, checks for proper firmware and/or calibration status of the gas monitors is performed, additionally or alternatively, by the gas station component.

Further in the example of FIG. 1, the gas station provides support for “bump” and “cal” operations. As used herein, a bump operation includes any type of rationality check to ensure that the gas monitor is responsive, for example providing the gas monitor with a plug of a specified gas, and/or a step change in a specified gas, and observing the monitor to ensure that an expected response occurs. As used herein, a calibration operation includes any type of adjustment to the fundamental detection relationship for the gas monitor, for example including updates to any A/D processing (e.g., filters, cut-offs, offsets, scaling, etc.), changes to any models utilized, and/or changes to any coefficients utilized by such models. In certain embodiments, calibration changes may be made in response to any changes in the base models, sensor drift, sensor wear, other changes in the gas monitor over time and/or utilization, and/or changes in the facility—for example facility changes that may indicate that a different model or model settings should be utilized (e.g., facility changes may lead to distinct environments that may affect gas monitoring operations, for example changes in airflow patterns, ambient temperature, background gas constituency, etc.). In certain embodiments, the gas station provides full physical support for bump/cal operations, including for example flowing selected amounts of selected gases through the sensor to support operations. In certain embodiments, the gas station can support multiple monitors mounted thereon, for example at one of a number of mounting locations. In certain embodiments, for example depending upon the number and mix of sensors at the location, the mounting locations may be of varying support configurations—for example a gas station may have five docking locations for supporting a single-gas detection monitor, and two additional docking locations for supporting a four-gas detection monitor. The example gas station performs the bump and cal operations (potentially with support from the data manager as set forth preceding), which is performed automatically in response to the docking operation and frees up the monitoring device for the user (e.g., to store, begin a shift, place on a charger, etc.). In certain embodiments, the gas station may optionally charge the monitoring class device while docked, may have separate charging docks, may have a charging area (e.g., a flat area configured for wireless charging), and/or charging operations for monitoring devices may be provided separately (e.g., a charger positioned in a storage area, etc.).

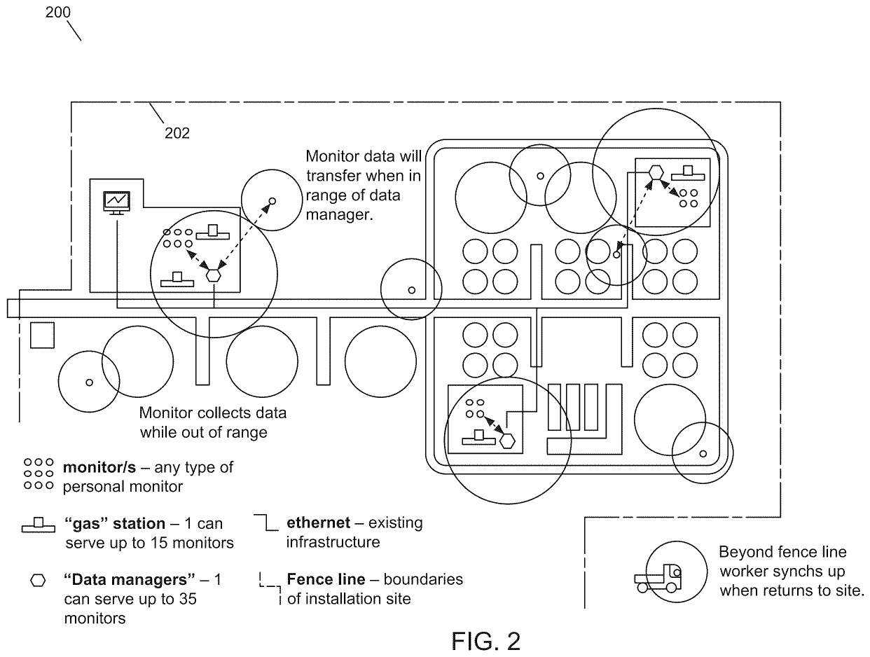

Referencing FIG. 2, another example gas monitoring system 200 includes a facility having a number of gas monitors that have a short range low power network capability. The example gas monitoring system utilizes 100 personal monitors for personnel operating the facility, supported by three data managers and four gas stations, and it should be understood that there is no limitation as to how many monitors a gas station can support. In the example of FIG. 2, the selected data managers are provided according to, for example, the typical locations of personnel to ensure they are in proximity of a data manager during a shift. In the example of FIG. 2, the selected gas stations are provided for sufficient operator support so operational impact of bump/cal operations is minimized. In the example of FIG. 2, two gas stations are co-located in the upper left portion, based on for example the level of operator activity at that location. In the example of FIG. 2, each one of the gas monitors has low power network capability, and utilizes the data manager to tie into the network and communicate with external devices. In the example of FIG. 2, the data managers utilize an ethernet network for on-site communication and coordination, and a lead one, or all, of the data managers can further communicate with external devices utilizing an internet connection, cellular, satellite, or the like. In the example of FIG. 2, monitoring devices in proximity to a data manager can transfer monitor data, receive firmware, receive messages and alerts, or the like from the data manager. In the example of FIG. 2, monitoring devices outside of the proximity of a data manager can link in to the low power network through other monitoring devices in proximity to the device, and/or through anchors or repeaters. In certain embodiments, activity on the low power network for a monitoring device may be limited depending upon how it is connected to the network—for example peer connections (e.g., between monitoring devices) may be limited to alerts, status notifications, passing priority messages, or the like, with other operations such as communicating monitored data, updating firmware, or the like limited to connections with a data manager or as otherwise described herein. A fence line 202 is shown indicating the boundaries of the installation site.

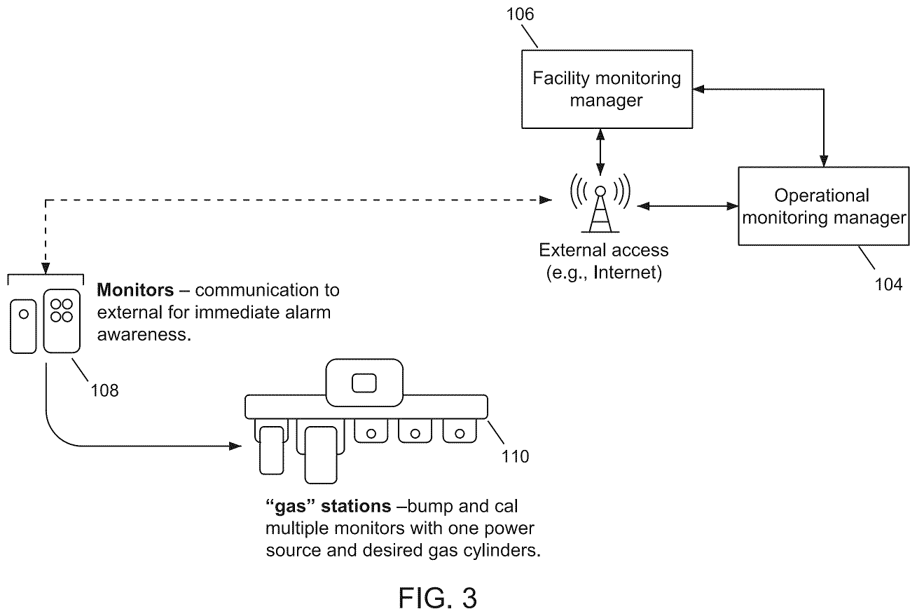

Referencing FIG. 3, it can be seen that a gas station 110 can reach external devices without a data manager, for example where one or more of the monitoring devices 108 has sufficient capability to communicate externally (e.g., using cellular or satellite communications). In certain embodiments, the system of FIG. 3 can provide numerous benefits of the gas station as disclosed herein, without requiring the installation of a data manager in proximity to the gas station.

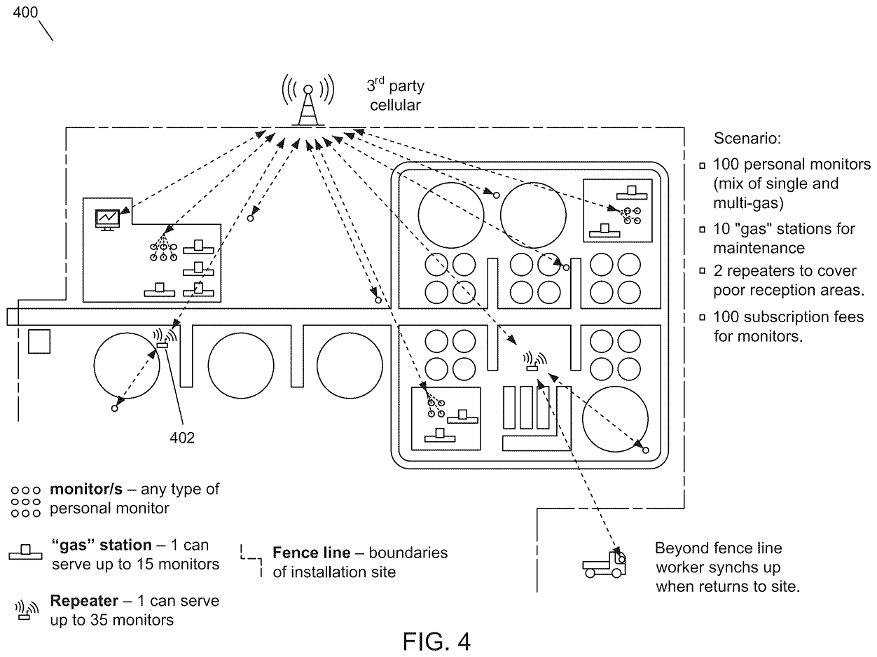

Referencing FIG. 4, another example gas monitoring system 400 is schematically depicted. In the example of FIG. 4, one or more monitoring devices in the system are capable of high power communications, for example utilizing cellular and/or satellite communications. The example of FIG. 4 utilizes repeaters 402 to provide connectivity in challenging areas, and to support gas stations. In some embodiments, the repeater 402 serves as a cellular booster. It should be understood that there is no limit to the number of monitors a repeater 402 can support. A system can be built with high capability monitoring devices as depicted in FIG. 4, and/or a system such as that depicted in FIG. 2 can be configured to utilize high capability monitoring devices as available within the system, while supporting low capability monitoring devices and the consequent benefits thereof (e.g., lower cost, reduced operational complexity, reduced power consumption). In some embodiments, one or more subscription fees are associated with the gas monitoring system or components thereof (e.g., on each monitor, on a group of monitors, on each gas station, on the system). The one or more subscription fees can appropriately attribute cost to the beneficiaries of the gas monitoring and/or provide an accounting lever to support efficient operation and/or iterative improvement of operations.

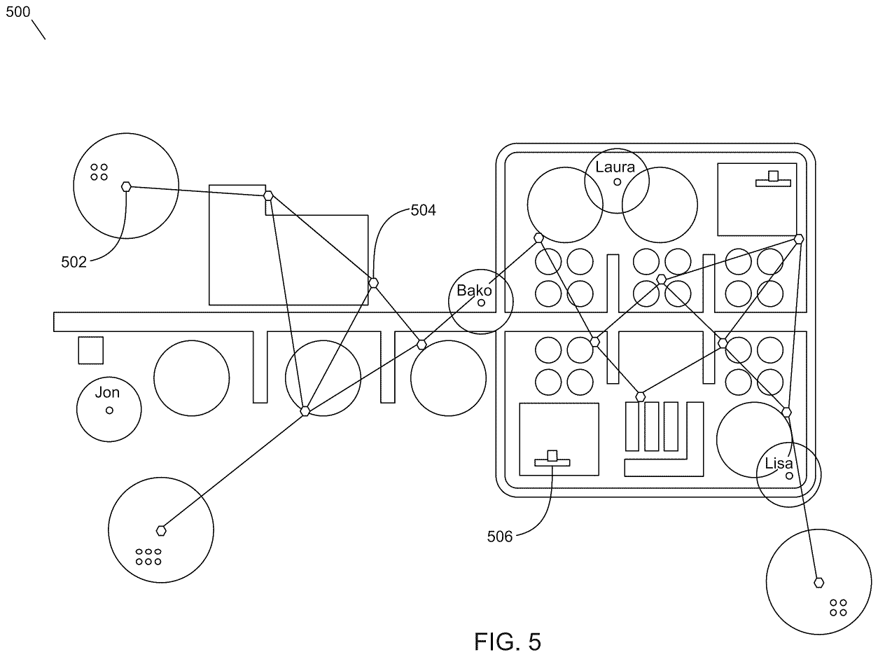

Referencing FIG. 5, another example gas monitoring system 500 is schematically depicted. The example of FIG. 5 includes the utilization of a number of low power anchors 504 that extend connectivity of the low power network as desired in the facility, including to create connectivity in challenging locations. The anchors form a static portion of a mesh network including the data managers 502, and the gas monitors, and optionally the gas stations 506, and have sufficiently low power requirements that a typical monitor in a convenient format can operate from battery for extended periods, up to about three years, without requiring a charge or battery change. In certain embodiments, one or more of the anchors 504 can be provided with one or more sensors, and can be used to rapidly provide a fence line or monitored region. For example, a notification or alarm may be generated, and optionally transmitted to other devices, if an instrument leaves a perimeter established by one or more of the data manager or an anchor. In certain embodiments, the anchors can be utilized to improve the available position information about specific gas monitors. In some embodiments, an operator or connected device location, such as a three-dimensional location (e.g., latitude, longitude, and height) may be determined from the placement of anchors in communication with operators/devices.

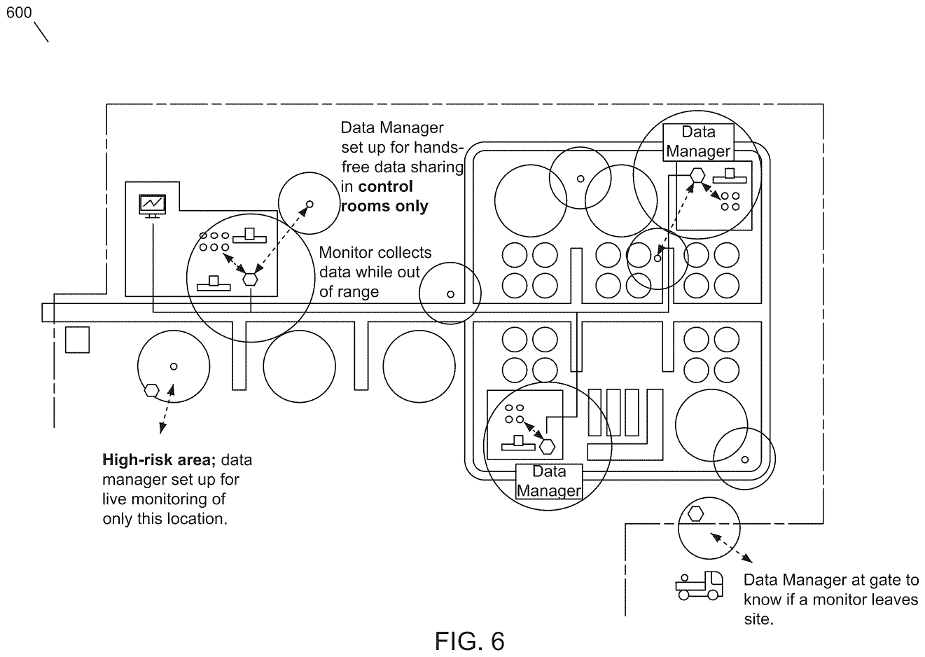

Referencing FIG. 6, another example gas monitoring system 600 is schematically depicted. The example of FIG. 6 includes a data manager positioned in a sensitive location, for example to ensure that connectivity in the area is successful, and to provide high rate data communication at the location—for example allowing for collection of faster sampling data, higher resolution data, keeping longer data sequences (e.g., a monitor out-of-connection or only connected through other low power network devices may be limited in the amount of data that can be buffered or saved), and/or supporting low latency analysis that may depend on a larger set of data than is typically provided by a monitor operating in an area that does not have constant high speed connectivity. In some embodiments of the example gas monitoring system 600, an anchor may be used in place of the data manager to provide connectivity and communication.

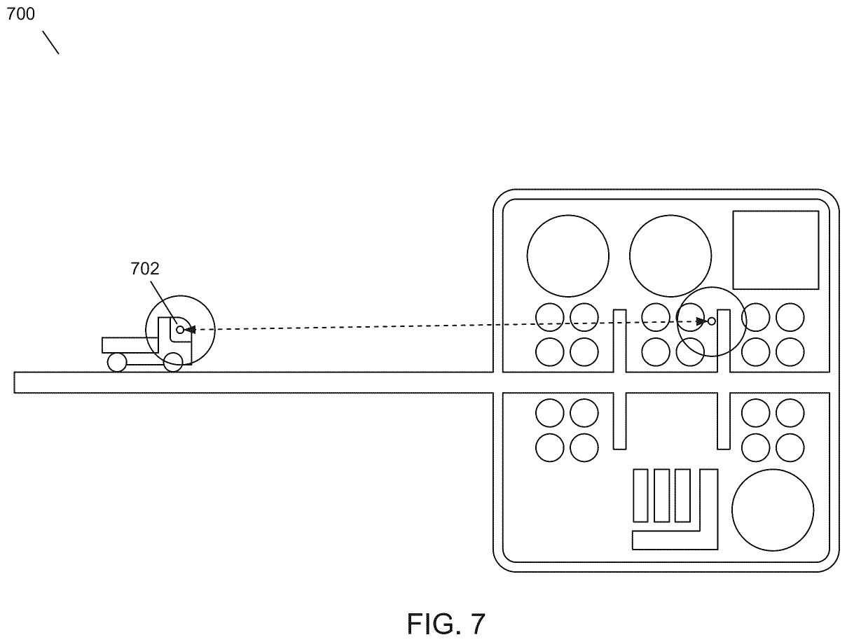

Referencing FIG. 7, an example gas monitoring system 700 is schematically depicted, with a monitoring device 702 currently depicted off location. In the example of FIG. 7, when the isolated monitoring device comes into communication range with a data manager, the data from the monitoring device is downloaded, and firmware and calibration data is confirmed and/or updated. In certain embodiments, the isolated monitoring device may also provide a notification to the user if a more involved operation is involved, for example if the firmware needs to be updated, the device requires a bump or calibration test, and/or if the device needs service or should be checked.

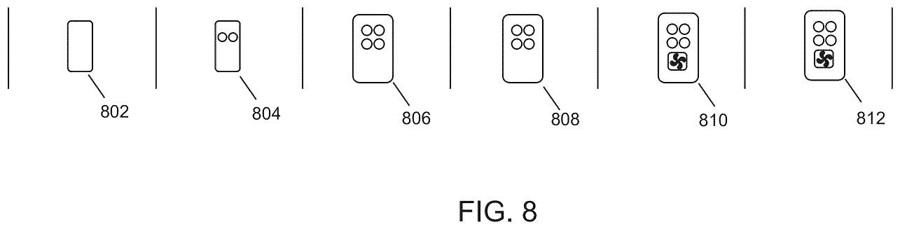

Referencing FIG. 8, a number of example monitoring devices are schematically depicted. Devices can range from a single gas sensing device to a multi-gas sensing device, and can include passive or active sensors (e.g., forced flow sensors). In certain embodiments, monitoring devices may include other types of sensors as indicated preceding. In certain embodiments, expensive or complex gas monitoring devices may also further include higher power network connectivity options (e.g., cellular and/or satellite), given the lower cost ratio of providing such connectivity in view of the cost of the device overall. However, even where expensive or complex gas monitoring devices are present, those devices may nevertheless not include higher power network connectivity (e.g., for security reasons, and/or to minimize power consumption), and/or the high power communications may be reduced where possible to reduce power consumption, and accordingly such devices provide additional capability in certain embodiments, and still benefit from a gas monitoring system having multiple device classes. Monitor 802 does not detect gas, gas monitor 804 detects a single gas, gas monitor 806 is a portable diffusion monitor configured to detect up to 4 gases, gas monitor 808 is a portable diffusion monitor configured to detect up to 5 gases, including exotic gases, gas monitor 810 is an aspirated gas monitor configured to detect up to 4 gases, and gas monitor 812 is an aspirated gas monitor configured to detect exotic gases.

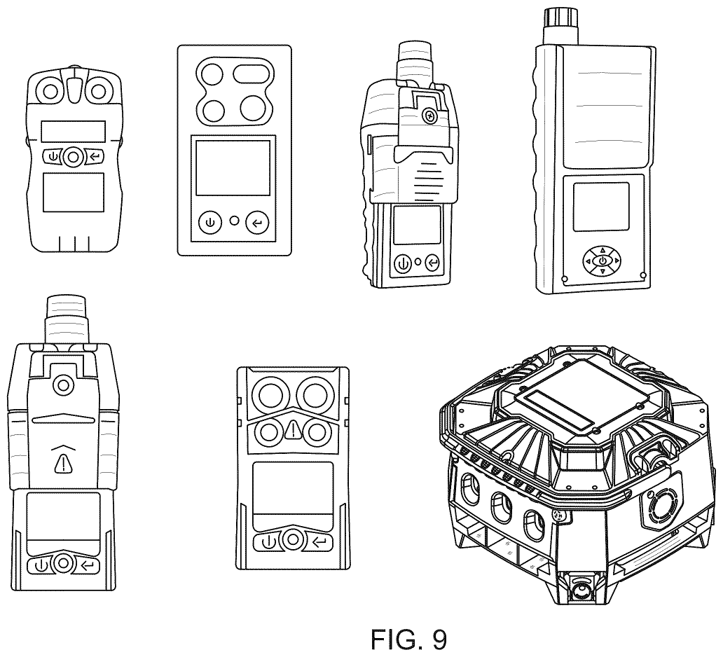

Referencing FIG. 9, example and non-limiting gas monitoring devices compatible with systems herein are schematically depicted. Depending upon the system configuration and the current operations, such devices may operate as a monitoring device, as an anchor, and/or as a data manager.

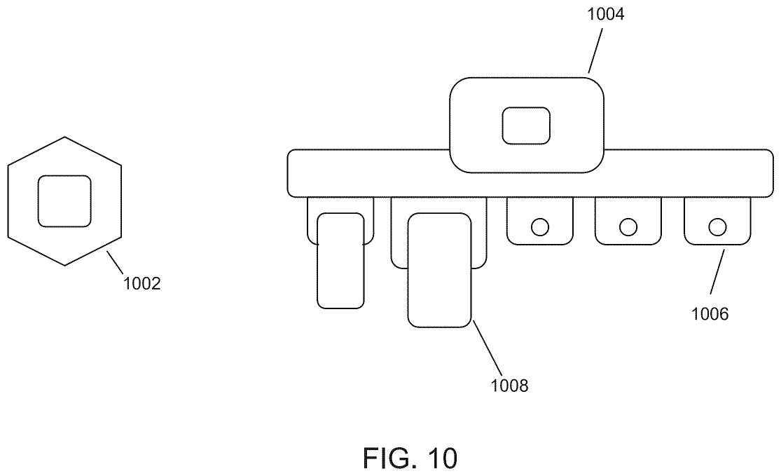

Referencing FIG. 10, an example data manager component 1002 (left) is schematically depicted, and shown as in the preceding figures. FIG. 10 further depicts a gas station component 1004, having two different monitor components 1008 docked thereon at gas monitor physical interfaces 1006, depicted as shown in the preceding figures. The example physical interfaces 1006 allow the gas station to provide power to the docked gas monitor, to communicate with the gas monitor (e.g., to download data from the gas monitor, to perform diagnostics and/or retrieve diagnostic data, to perform calibration operations, and/or to update firmware and/or calibrations of the gas monitor), and/or to provide gas flow to the gas monitor (e.g., providing a selected gas to a sensing element of the gas monitor, for example to perform a bump test and/or a response characterization of the sensing element to a selected gas at a selected concentration).

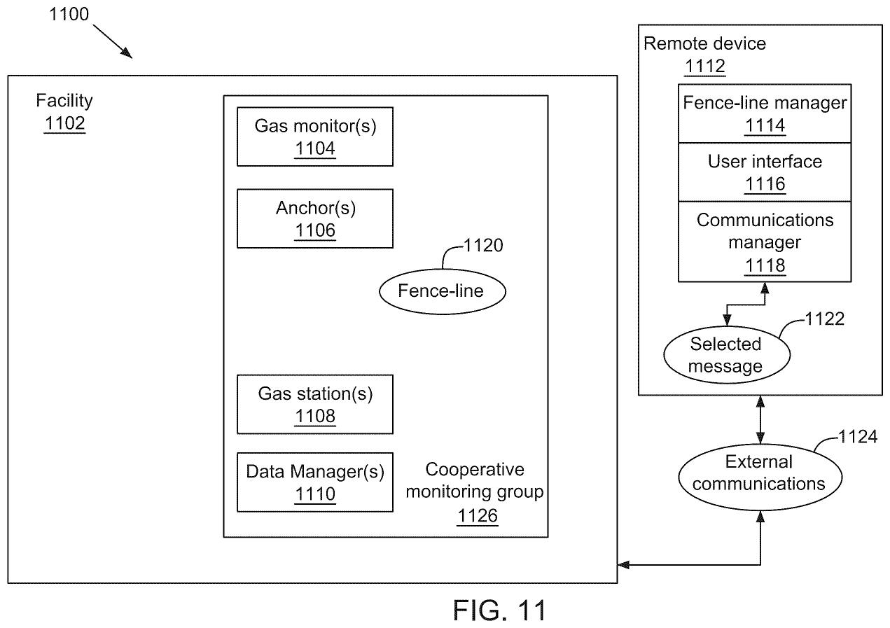

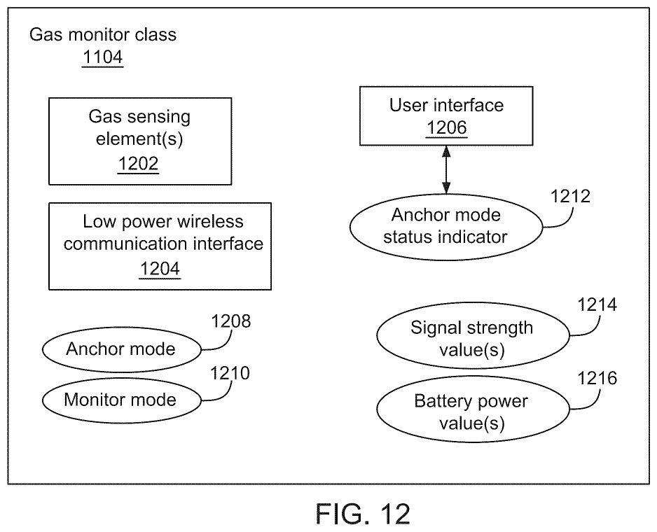

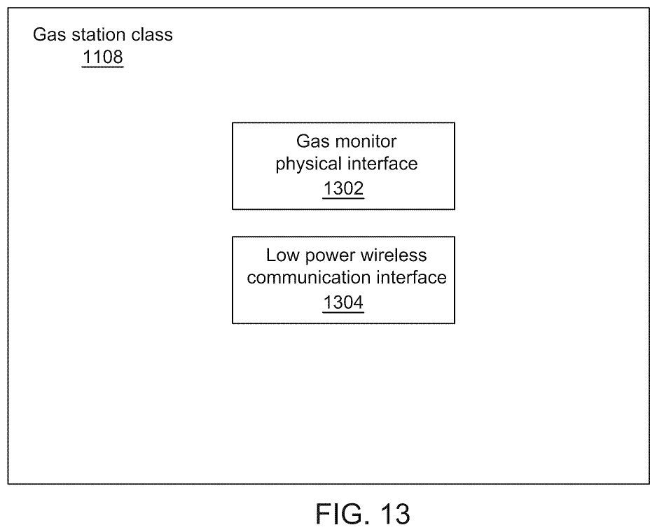

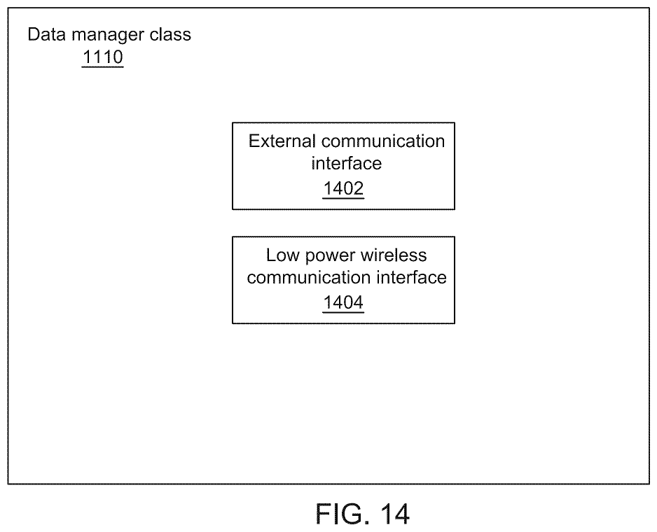

Referencing FIG. 11, an example system 1100 schematically depicts aspects of embodiments of the present disclosure. The example system 1100 includes a cooperative monitoring group 1126 including group members from a gas monitor class 1104, gas station class 1108, and data manager class 1110. In certain embodiments, the system 1100 further includes members from an anchor class 1106, and/or one or more fence-lines 1120. In example systems, the fence-line 1120 may be formed from a number of anchors 1106 and/or gas monitors 1104 operating as an anchor. The cooperative monitoring group 1126 is deployed at a facility 1102 having a network of end points. The end points can be of a gas monitor class 1104 (e.g., a gas monitor), a gas station class 1108 (e.g., a gas station), a data manager class 1110 (e.g., a data manager), or an anchor class. Gas monitor class 1104 includes a gas sensing element 1202, a low power wireless communication interface 1204, and a user interface 1206, as seen in FIG. 12. Gas stations from the gas station class 1108 include at least one gas monitor physical interface 1302 and a wireless communication interface 1304, as seen in FIG. 13. Data managers from the data manager class 1110 include an external communication interface 1402 and a low power wireless communication interface 1404, as seen in FIG. 14. The cooperative monitoring group 1126 can operate as a mesh network in some embodiments. External communications 1124 of the cooperative monitoring group 1126 from end points of the gas station class and the gas monitor class can be routed through end points of the data manager class, which provides for reduced infrastructure requirements to deploy the cooperative monitoring group 1126, as gas monitors 1104 utilizing only low power wireless communications in a mesh network can be lighter weight and operate much longer without requiring a large battery, and as gas stations 1108 wirelessly coupled to the data manager 1110 can be positioned anywhere within communicative reach of the data manager 1110 (e.g., directly to the data manager 1110, and/or chained through the mesh network utilizing anchors) without supporting infrastructure such as ethernet or other wired network communications. External communications 1124 can include communications between remote devices 1112 and the facility 1102. In embodiments, remote devices 1112 can include a fence-line manager 1114, a user interface 1116, and a communications manager 1118. Remote devices 1112 include remote computing devices, mobile devices, a cloud server, or the like.

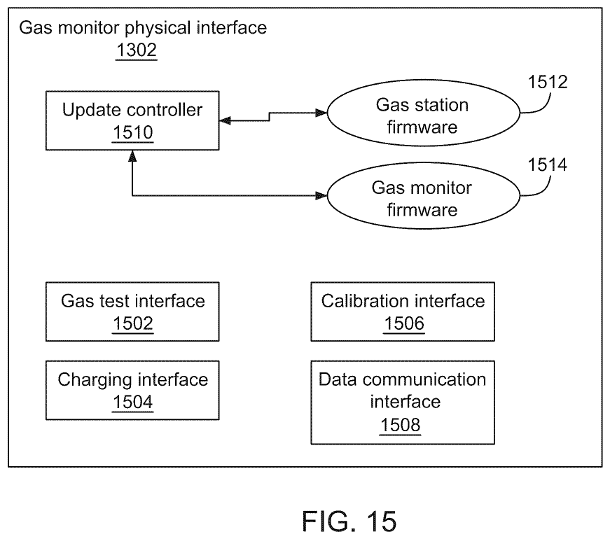

FIG. 15 depicts an example gas monitor physical interface 1302 of the gas station class 1108 that includes a gas test interface 1502, a charging interface 1504, a calibration interface 1506, and a data communication interface 1508. An update controller 1510 updates gas station firmware 1512 via external communications 1124. The update controller 1510 also updates the firmware of the gas monitor 1514 in response to the gas monitor class 1104 being coupled to the gas monitor physical interface 1302.

In other embodiments, the gas monitor physical interface 1302 does not include a gas test interface. One of skill in the art, having the benefit of the present disclosure and information ordinarily available about a contemplated system, can readily determine scenarios in which the gas test interface is not necessary (e.g., bump/cal operations are handled in a location remote from the gas station).

In an embodiment of the present disclosure, the end points of the cooperative monitoring group 1126 further include at least one end point from an anchor class. Anchors 1106 include a wireless communication interface and operate as a node in the mesh network. Anchors 1106 can be positioned at a determined position and include a battery to provide normal operation of the anchor 1106 for an extended duration, such as at least one year. External communications 1124 of the cooperative monitoring group 1126 from the end point(s) of anchor(s) 1106 are routed through end points of the data manager(s).

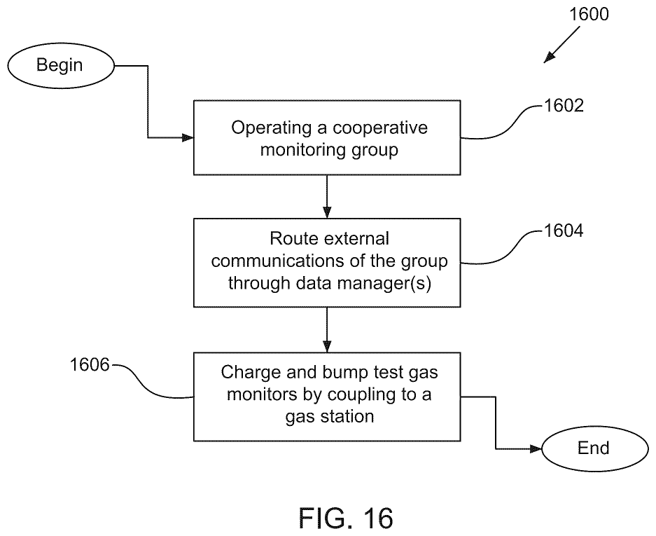

Referencing FIG. 16, an example procedure 1600 is depicted. The example procedure 1600 includes an operation 1602 of operating a cooperative monitoring group. The procedure 1600 includes an operation 1604 to route external communications of the group through data manager(s). The procedure includes an operation 1606 to charge and bump test gas monitors by coupling to a gas station.

In certain embodiments, the example procedure 1600 further includes an operation of extending a range of the mesh network utilizing an anchor of the cooperative monitoring group. The procedure 1600 may further include an operation of ensuring connectivity at a location of the facility utilizing an anchor of the cooperative monitoring group. The procedure 1600 may also include performing at least one operation of tracking locations of the plurality of gas monitors at the facility or tracking compliance of the plurality of gas monitors at the facility. The procedure 1600 may include an operation to route the alert from at least one of the plurality of gas monitors to a remote device through the at least one data manager.

In an embodiment of the present disclosure, procedure 1600 further includes an operation to update firmware of the gas station utilizing low power wireless communication between the gas station and the at least one data manager. The procedure 1600 may include an operation to update at least one of a calibration of at least one of the plurality of gas monitors or a firmware of at least one of the plurality of gas monitors in response to a coupling of the at least one of the plurality of gas monitors to the gas station.

The procedure 1600 may include an operation to perform a bump test on at least one of the plurality of gas monitors in response to a coupling of the at least one of the plurality of gas monitors to the gas station.

In some embodiments, the gas monitor class 1104 can operate in a number of modes. In one embodiment, it operates in a monitor mode 1210 while in another embodiment, it may operate in an anchor mode 1208. In anchor mode 1208, it may operate from a fixed position, such as mounted on a wall, and can operate as a node in the mesh network. In some examples, the gas monitor class 1104 operates in anchor mode 1208 in response to at least one of a selection on the gas monitor by a user, a selection in a mobile application by a user, or a selection in a user interface 1116 on a remote device 1112 or in a user interface 1206 on the gas monitor. The gas monitor publishes an anchor mode status indicator 1212 to other gas monitors on the mesh network. For example, a user may select the anchor mode on the gas monitor 1104, and hang the gas monitor 1104 near the entrance of a confined space or other risk area, providing both gas detection at the location, and preserving connectivity between another gas monitor (e.g., used by an operator entering the confined space) and the rest of the network, including back through a data manager and to a remote device (e.g., allowing managers, safety personnel, and/or any other supporting personnel to monitor the confined space operations). The fixed position may be a known position, determined by the gas monitor (e.g., using strength of signal operations), and/or specified by the user (e.g., at the time of engaging the anchor mode). In certain embodiments, the gas monitor 1104 may be operated in the anchor mode without being at a fixed position (e.g., associated with a user, mobile equipment, moved during anchoring operations, etc.). In certain embodiments, the gas monitor in the anchoring mode may continue to monitor gases using a sensing element of the gas monitor, and/or the gas monitor in the anchoring mode may stop monitoring gases (e.g., to dedicate computing power and/or battery power of the gas monitor to anchoring operations, such as relaying communications of other devices, extending the range of the mesh network, and/or bridging between different groups of monitors of the mesh network).

An example gas monitor monitors at least one gas constituent at the fixed position. A person of skill in the art having the benefit of this disclosure would understand when the gas monitor should operate in anchor mode versus monitor mode. For example, a worker carrying multiple gas monitors to a central location may choose to keep multiple monitors on their person operating in monitor mode as they inspect the central location, then as they move on to another area, they may choose to leave a monitor behind switched from monitor mode to anchor mode to extend the low power network as they move further away from the central location. Conversely, a worker in a remote location having connection through a gas monitor operating as an anchor to a low power network may require additional monitors for a particular operation. The worker would understand that they can readily switch the gas monitor from anchor mode to monitor mode to begin or resume monitoring operations, and/or to stop anchoring operations.

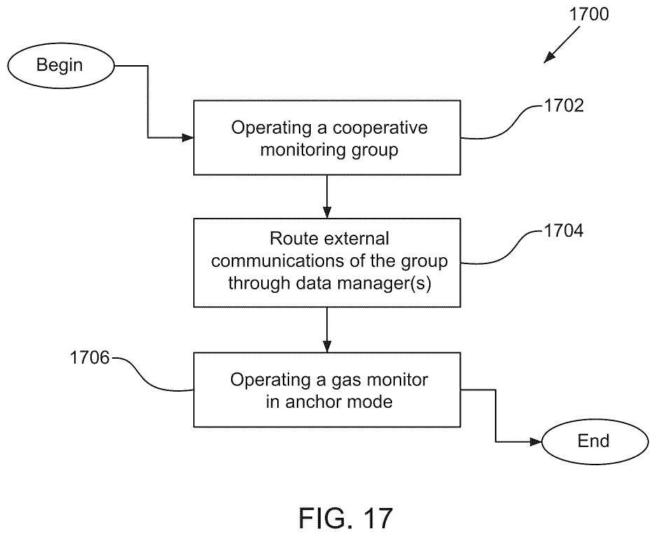

In an embodiment of the present disclosure, the example procedure 1700 as seen in FIG. 17 includes an operation 1702 to operate a cooperative monitoring group. Procedure 1700 includes an operation 1704 to route external communications of the group through data manager(s). Procedure 1700 includes an operation 1706 to operate a gas monitor in anchor mode.

In certain embodiments, the example procedure 1700 includes an operation to operate the at least one of the plurality of gas monitors in the anchor mode at a fixed position. In certain embodiments, the procedure 1600 further includes an operation to monitor at least one gas constituent at the fixed position. In other embodiments, procedure 1600 further includes publishing anchor mode status indicator to other gas monitors on the mesh network.

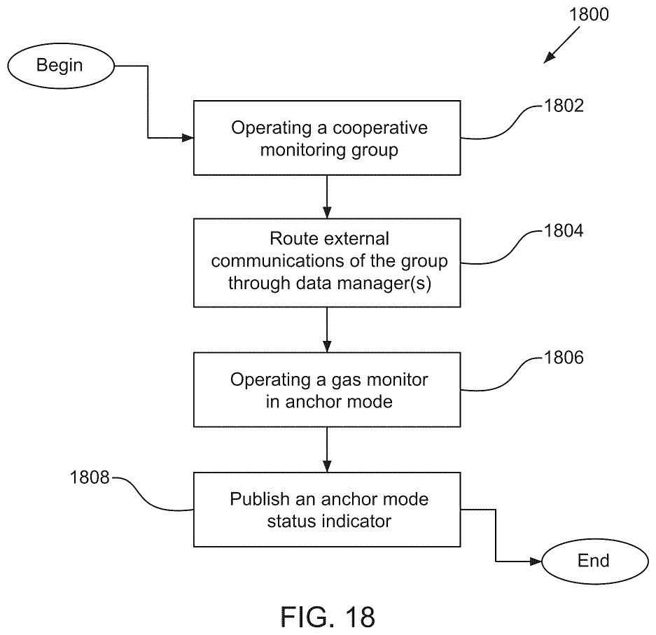

Referring to FIG. 18, an example procedure 1800 includes an operation 1802 to operate a cooperative monitoring group. Procedure 1800 includes an operation 1804 to route external communications of the group through data manager(s). Procedure 1800 includes an operation 1806 to operate a gas monitor in anchor mode. Procedure 1800 includes an operation 1808 to publish an anchor mode status indicator.

In certain embodiments, the procedure 1700 further includes selecting the anchor mode on the at least one of the plurality of gas monitors. In other embodiments, the procedure 1700 further includes selecting the anchor mode on a mobile application. In certain embodiments, the procedure 1700 includes selecting the anchor mode on a remote device.

In an embodiment of the present disclosure, a communications manager 1118 is configured to provide a selected message 1122 to a selected group of the plurality of gas monitors 1902. Utilizing a communications manager has several benefits including: avoiding SMS, being able to easily trigger a peer alarm (e.g., to selected devices, groups, location-based), readily triggering an alarm for entry to a zone or space, or easily send a notification to avoid a worker inadvertently leaving the facility with device. In certain embodiments, the communications manager allows for other personnel to be notified of, to direct and/or approve, and/or to view alarms, alerts, and/or other selected messages 1122. Numerous other benefits may also be present.

For example, gas monitors operating in a confined space may receive a periodic message to audit the operation of their personal alarm systems. In some embodiments, the communications manager 1118 is positioned on a remote device 1112. In the embodiment, each wireless communication interface (e.g., of the gas monitor, data manager and gas station) includes a low power wireless interface 1204. In an example, the selected group of the plurality of the gas monitors is a geofenced group 1904 of the gas monitors. For example, messages may be directed in particular to just those geofenced devices such as to provide additional information regarding procedures for operations of cooperative monitoring groups within the geofence, and/or to a group of devices selected for any reason (e.g., proximity to a hazard, in the path of an emerging or evolving hazard, devices associated with evacuating workers, to devices associated with selected workers, to devices having a certain sensor type or calibration settings, and/or to devices having a physical characteristic such as low remaining battery power).

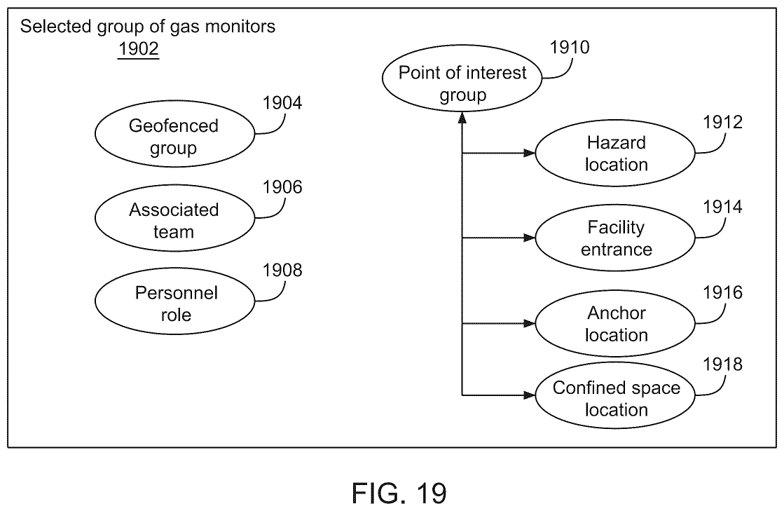

Referring to FIG. 19, the selected group of the plurality of gas monitors 1902 is a group of the gas monitors within a selected range of a point of interest, which may also be referred to as a point of interest group 1910. The point of interest could be a hazard location 1912, a facility entrance 1914, an anchor location 1916, and/or a confined space location 1918. The selected group of monitors is associated with a selected team 1906 (a.k.a. associated team 1906) and/or a personnel role 1908.

The example communications manager 1118 is depicted as positioned on the remote device 1112 for clarity of the present description. Additionally or alternatively, the communications manager 1118 may be positioned, in whole or part, on one or more devices in the system such as the remote device 1112, gas monitor(s) 1104, data manager(s) 1110, gas station(s) 1108, and/or anchor(s) 1106, and/or distributed among one or more of these. In certain embodiments, for example where portions of the communications manager 1118 are positioned on an intermediate device (e.g., the data manager 1110, gas station 1108, and/or anchor 1106), the selected message 1122 can initiate at one of the plurality of gas monitors or at a remote device 1112.

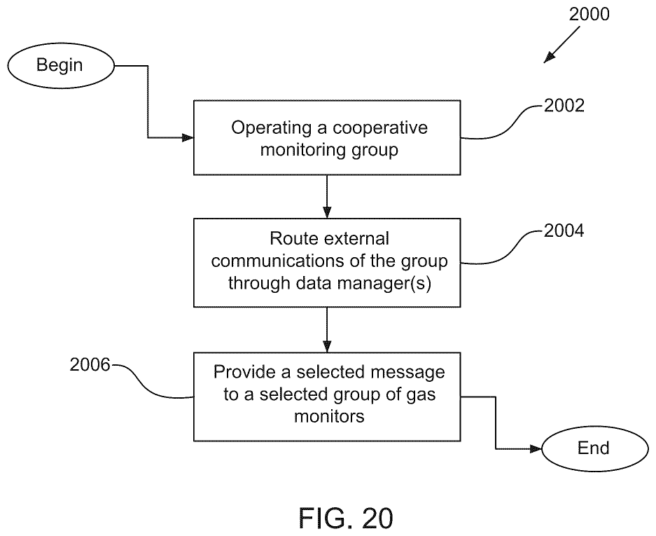

As seen in FIG. 20, an example procedure 2000 includes an operation 2002 to operate a cooperative monitoring group. The procedure includes an operation 2004 to route external communications of the cooperative monitoring group through at least one data manager. The procedure includes an operation 2006 to provide a selected message to a selected group of the gas monitors. The procedure optionally includes an operation to initiate the selected message at a gas monitor or at a remote device.

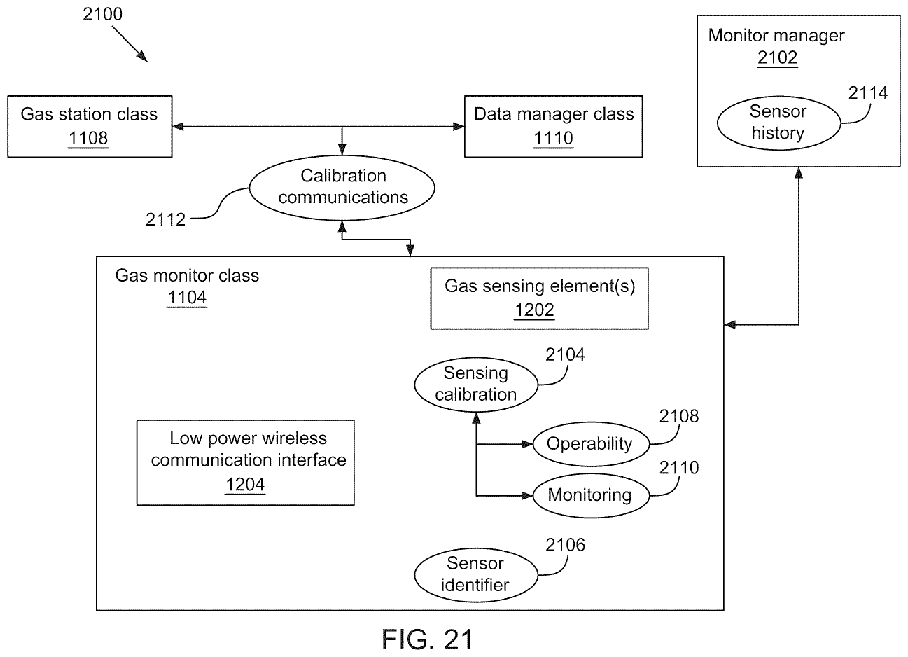

Turning to FIG. 21, an example system 2100 that includes distribution of instrument calibration operations is shown. In the example system 2100, the components of a cooperative monitoring group (e.g. gas station class 1108, gas monitor class 1104, data manager class 1110) can share calibration communications with each other to ensure that one or all members of the group are appropriately calibrated. Instrument calibrations can be important during or after manufacturing, in the course of routine or acute maintenance, and for routine and/or mission critical service updates. A person of skill in the art would be able to identify an appropriate calibration as one that is in response to, or otherwise accounts for, a maintenance duration, a change in firmware, a change in cooperative monitoring group make-up, a change in operation/task, a change in facility or monitoring location, a change in weather, or the like. A monitor manager 2102, which retains a sensor history 2114, can be used to manage or simply check the status of calibration(s).

The example system 2100 includes a gas monitor class 1104 having a gas sensing element 1202, a wireless communication interface 1204, a sensor calibration 2104 (a.k.a. sensing calibration 2104), and a sensor identifier 2106. The sensor calibration 2104 also includes an operability 2108 calibration. The system 2100 also includes a gas station class 1108 which includes a gas monitor physical interface 1302 and a wireless communication interface 1304. The system 2100 also includes a data manager class 1110 which includes a wireless communication interface 1404 and an external communication interface 1402. The data manager class 1110 implements external communications 1124 between a remote device 1112 and the gas monitor class 1104 and between the remote device 1112 and the gas station class 1108. At least one of the gas station class 1108 or the data manager class 1110 updates the sensor calibration 2104 to a monitoring calibration 2110. In the example system 2100, calibration communications 2112 are routed between the gas monitor class 1104, gas station class 1108, and the data manager class 1110.

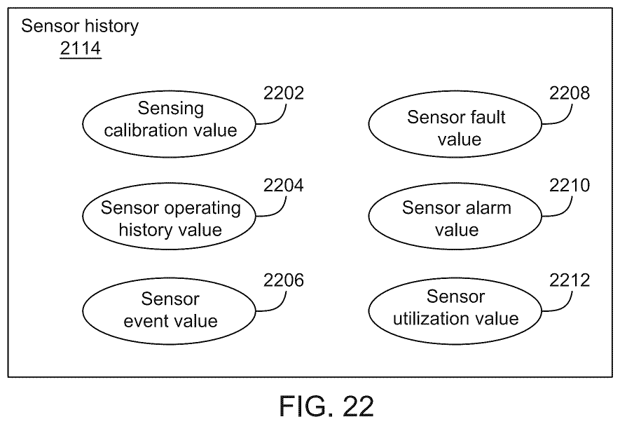

In the system 2100, the gas sensing element 1202 includes a computing device to host the sensing calibration value 2202. In embodiments, the sensor identifier 2106 is associated with the computing device, and the monitor manager 2102 is configured to track a sensor history 2114 associated with the sensor identifier 2106. Referring now to FIG. 22, the sensor history 2114 can include any value measured by, transmitted by, or transmitted to the gas sensing element 1202. Sensor history 2114 can include many different values, such as for example, a sensor calibration value 2202 (e.g., how many times the sensor has been calibrated, what concentration of gas it was calibrated against, how much drift the sensor experienced from the last calibration), a sensor operating history value 2204 (e.g., how long the sensor has been in service, how often the sensor detected gas above a threshold level, how many times the sensor detected an out of range level), a sensor event value 2206 (e.g., how many alarms were triggered by the sensor's measurement, how many times two sensors each detected an out of range level at the same time), a sensor fault value 2208 (e.g., how often a fault was detected in operation or calibration), a sensor alarm value 2210 (e.g., how many times a sensor went into alarm, how many times two or more sensors went into alarm for different gases), or a sensor utilization value 2212 (e.g., total service hours for a sensor). The monitor manager 2102 having access to all of these values enables robust management of each class of devices and communications, such as calibration communications 2112, between them.

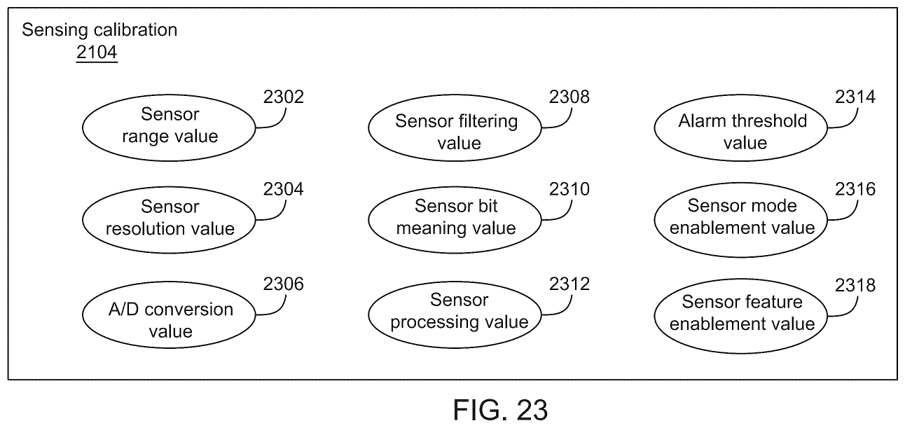

In embodiments, and referring now to FIG. 23, the sensing calibration 2104 includes at least one of a sensor range value 2302 or a sensor resolution value 2304. The sensor range value 2302 can refer to a maximum sensed value of a sensor, a minimum sensed value of a sensor, the range of sensed values, the range of sensed values in light of a particular impacting factor (e.g., temperature, light, power), the accuracy of the sensor over an operational period, compensation values utilized for the sensor over the detected range, saturation values for the sensor (and/or actions taken at saturation, such as waiting times, filtering changes, reset values, etc.). The sensor resolution value 2304 refers to the smallest change the sensor can detect and/or that the sensor will output.

In some embodiments, the sensor calibration 2104 includes one or more of a sensor analog-to-digital conversion value 2306 (e.g., a sample rate, signal-to-noise ratio, resolution, input range, conversion equations and/or values therefore, gains, etc.), a sensor filtering value 2308 (e.g., a low-pass cutoff, a high-pass cutoff, a rolloff/attenuation, time constants, and/or response factors for filtering such as changes based on the detected value, sensor age or exposure history, etc.), a sensor bit meaning value 2310 (e.g., 8-bit, 12-bit, 16-bit, high resolution, low resolution, reserved bit values utilized for diagnostics, reporting of sensor states, and/or fault values, etc.), a sensor processing value 2312 (e.g., an amplification, a filtering, an algorithm for identifying gas, a correlation algorithm, etc.), an alarm threshold value 2314 (e.g., a regulatory threshold, a user threshold, a company threshold, an industry threshold, and/or a standardized or 3^{rd} party threshold), a sensor mode enablement value 2316 (e.g., monitoring vs. anchor, detection regime, response curve to be utilized, compensation operations to be performed based on detected conditions, etc.), or a sensor feature enablement value 2318 (e.g., sensitivity, resolution, range, accuracy, response time, diagnostic features, correlated gas constituent detection operations, etc.).

The disclosure herein provides numerous benefits with respect to fence-line challenges. In some embodiments of the disclosure, gas monitors can also serve as anchors. An example system 1100 includes a cooperative monitoring group 1126 of gas monitors and a fence-line 1120 including a plurality of gas monitors at selected locations, such as a fixed location or a mobile location. The selected location is determined in response to at least one signal strength value 1214. For example, to ensure that the fence-line operates as intended, gas monitors are distributed at intervals which can be determined using signal strength and/or position from a central location. In this example, monitors of the fence-line 1120 in a central part may have the strongest signal strength as measured from a point in the central part, while the signal strength as measured in the central part from monitors disposed on the fence-line 1120 further from central part is weaker. A lowest signal strength from a fixed point can be established as limiting for membership in the fence-line 1120. In certain embodiments, the monitors and/or anchors used on the fence-line can chain back to the central connection to the main mesh network, with signal strengths utilized to determine positions, detect issues with the fence-line, and/or improve or optimize power utilization to support communication operations.

The cooperative monitoring group 1126 includes a network of end points, the end points including at least one end point from each class selected from a gas monitor class 1104, a gas station class 1108, and a data manager class 1110. In the gas monitor class 1104, each gas monitor includes a gas sensing element and a wireless communication interface. In the gas station class 1108, each gas station comprising at least one gas monitor physical interface, and a wireless communication interface. In the data manager class 1110, each data manager comprising a wireless communication interface and an external communication interface.

The cooperative monitoring group 1126 is configured to operate as a mesh network, and external communications 1124 of the cooperative monitoring group 1126 from end points of the gas station class 1108 and the gas monitor class 1104 are routed through end points of the data manager class 1110.

Each one of the plurality of gas monitors of the example system is configured to operate in at least one of a monitoring mode 1210 or an anchor mode 1208. A fence-line manager 1114 is configured to determine operating modes for the plurality of gas monitors in response to a battery power value 1216 for at least one of the plurality of gas monitors. For example, if the battery power value 1216 reaches a level that is approaching a lowest predetermined threshold, the fence-line manager 1114 may switch it from monitor mode 1210 to anchor mode 1208. The fence-line manager 1114 may also initiate additional actions such as notify personnel of the need to charge the monitor. The fence-line manager 1114 is configured to monitor the plurality of gas monitors as a fence-line group (or geofenced group 1904), determine a geofenced region in response to the selected locations, and monitor a geofenced region in response to the selected locations. For example, the geofenced region is defined by a number of monitors and anchors at fixed locations and gas monitors with a received signal strength at the fence-line manager above a certain threshold. In another example, the geofenced region includes actual geospatial coordinates with at least one boundary set by a fixed location of a gas monitor. In yet another example, the geofenced region is defined by a combination of gas monitors with a received signal strength at the fence-line manager above a certain threshold and actual geospatial coordinates.

In certain embodiments, the data manager class component manages firmware for the monitoring device class components. In certain embodiments, the data manager component interprets data from the monitoring device in response to planned conditions, for example the monitoring device entering proximity with the data manager and/or the monitoring device engaging with a dock on a gas station class component, and determines that the firmware on the monitoring device should be updated (e.g., checking versions or other indicators for the firmware on the device), and in response to determining the firmware should be updated performs operations to update the firmware. In certain embodiments, the firmware may be updated during operations where the monitoring device is known to be in a stable place and will not be relied upon for monitoring operations, for example during a dock on a gas station, while in a storage location, and/or during a time of day when the associated operator for the gas monitor (if applicable) does not work, or typically does not work.

Additionally or alternatively, the firmware update may include providing a notification from the monitor (e.g., an alert, light, displayed message, selected sound, etc.) that monitoring operations are temporarily unavailable during firmware update operations. In certain embodiments, for example in systems where the monitors do not have predictable downtime periods, and/or where it is undesirable that monitors have a downtime period, the data class manager component can download the new firmware in chunks, for example providing chunk sizes that are reasonable to download from the data manager during transient communication operations (e.g., within 10 seconds) between the data manager and the gas monitoring device. Each chunk can be downloaded and confirmed, or repeated as needed, until all of the chunks of the firmware update (or a portion of the firmware that can be implemented) are provided, and then later at a selected time the firmware update can be implemented. The final implementation of the firmware update can be commanded by the data manager directly (e.g., determining to flip the switch during a docking operation, expected downtime, when the monitor is in a selected location such as storage, etc.), or by a controller on the gas monitor, for example responding to a flag set by the data manager than a firmware update should be performed. In certain embodiments, the controller on the gas monitor can provide various feedback related to the firmware update, for example confirming the receipt of firmware download chunks, confirming the update of the firmware, and/or providing any messages or logs related to errors, failure to complete the update, and/or improper operation after the update.

In certain embodiments, the gas station includes a controller configured to perform various operations of the gas station, including for example performing bump tests, calibration operations, communicating with the data manager (e.g., to receive updated firmware, to confirm bump/cal data, etc.). In certain embodiments, the resulting data from the bump/cal operations is passed to the data manager from the gas station, and/or the data from the bump/cal operations may additionally be stored on the respective gas monitoring device and/or communicated to the data manager from the respective gas monitoring device. In certain embodiments, the controller on the gas station includes controlling gas flow from one or more test gases (e.g., controlling flow routing between various gas bottles and the docked monitor(s)) to docked gas monitor(s) to perform bump/cal operations, reporting on gas bottle levels, and/or providing information about faults, diagnostic operations, or the like related to the gas delivery system, docking and communication system, and/or related to charging operations (where applicable).

In certain embodiments, the interaction of the high power network connection to external devices, combined with a low power mesh network of devices on a gas monitoring system, combine to allow for a number of operations described following. An example operation includes a situational interaction operation with specific gas monitor(s) based on operating conditions within the facility. For example, alarm thresholds within the gas monitor may be adjusted according to the operating conditions. In a further example, a person performing a specific action that is visible to the system, for example according to specific procedures such as the completion of forms (e.g., a form completed for a confined space entry, a lockout/tagout operation, or the like, which may be electronically submitted and have fields therefore that can be interpreted by a controller on the data manager, by the facility monitoring manager, by the operational monitoring manager, and/or by the individual gas monitor for that operator.

In response to the operating conditions, the appropriate controller(s) may be configured to perform a situation interaction operation such as: commencing or stopping monitoring operations; adjusting an alarm threshold value (e.g., move from 5 ppm alarm to 25 ppm alarm; moving from a 1-second average based determination to a 3-second average based determination; etc.); adjusting an alarm response value (e.g., changing a location of alarm communications, changing alarm content such as selected lights, sounds, and/or messages, etc.); and/or adjusting a secondary parameter related to the gas monitoring system, such as an evacuation routing, adjusting a distance value for relating monitors at the facility (e.g., a system that utilizes 25 feet to consider those monitors to be related during nominal operations, switches to 10 feet to consider monitors to be related during high pressure operations on a tank having a highly toxic gas; and/or could include switching a facility relationship map from a first geofencing regime to a second geofencing regime—for example to change the risk assessment of the entire facility based on a global parameter such as “producing product A”, “producing product B”, “during Alert Type A”, “inclement weather preparation”, etc.). Without limitation to any other aspect of the present disclosure, example and non-limiting situational interaction operations include one or more operations such as: adjusting an allowable power consumption value for a device in the gas monitoring system; adjusting a monitoring operation; adjusting an alerting operation; adjusting an alert response operation; providing a selected notification to a selected external device; setting a flag to command a calibration and/or firmware update to a device (e.g., which may be used by a controller on the gas station and/or the data manager on a next docking event for the gas monitor); and/or setting a flag for a device to be turned in for analysis (e.g., which may be used anywhere in the system, for example the gas station may avoid performing bump/cal operations on such a device to avoid forensic complications, and/or the device may give the user a notice to turn it in to service or maintenance, etc.).

In certain embodiments, operations herein are performed to determine an operating condition for the facility, for a portion of the facility (e.g., an absolute portion such as “near the distillation column” or a relative portion such as “within 30 feet of gas monitor XYZ”), for a gas monitor, and/or for an operator associated with a gas monitor. Such operations may be performed by a controller (or controllers) in the system, including a controller positioned (at least in part) on the operational monitoring manager, the facility monitoring manager, a data manager, an anchor, a gas monitor, and/or the gas station. In certain embodiments, the respective operating condition is determined directly from data available to the system (e.g., users may check in and out to the facility, providing an electronic indication of whether they are present; the facility monitoring manager may provide information to the operational monitoring manager, such as a global operating state of the facility (e.g., RUN, SHUTDOWN, PROCESS A, STAGE B, etc.), one or more operating parameters for the facility and/or equipment thereof, or the like. In certain embodiments, the respective operating condition may be inferred and/or estimated based on other parameters (e.g., the calendar, weather events, number of personnel at the facility, etc.), and/or the respective operating condition may be explicitly set by and administrator or supervisor, and/or directly available system information such as information parsed from procedural data such as entry of a lockout/tagout procedure that is available to the facility monitoring manager or another controller in the system. In certain embodiments, the respective operating condition may be inferred from location of personnel, utilization of sensors to detect personnel conditions (e.g., the wearing of selected personal protective equipment, which may be determined based on utilization of smart equipment such as a smart respirator mask, and/or inferred from camera information). In certain embodiments, the type of operation, location of personnel, the hazards to be monitored, and the PPE worn by the operator, may all be utilized to determine the current threshold levels for alerts and alert responsive activity.

In certain embodiments, the gas monitoring system allows for specific messages and/or messaging/alerting/notification regimes to be provided in response to selected instruments within the gas monitoring system. Such messages can be sourced from any device in the system, for example from the facility monitoring manager, the operational monitoring manager, and/or one or more of the gas monitors, and can be provided to any device or selected group of devices within the gas monitoring system. For example, a message can be provided to all gas monitors in a particular area of the facility, within a certain distance from another gas monitor (e.g., a gas monitor that has an issue or an active alert), to a defined list of gas monitors (e.g., associated with a certain personnel group, regardless of location), to a defined list of devices, and/or to a contingent list of devices (e.g., all gas monitors that have been calibrated within the last 10 days). The messages can vary in priority and/or importance, with a selectively scheduled delivery such sending a message the next time each gas monitor enters a data manager zone, and/or can be utilized to override normal behavior—for example allowing a first high priority message to be propagated to all available devices right away (e.g., chaining on the low power network), and only providing a second lower priority message to be propagated to devices under non-disruptive conditions (e.g., to preserve power utilization for devices where the message is not urgent). In certain embodiments, a gas monitor user can send a message from their gas monitor to other gas monitors in the area, to the facility monitoring manager, and/or to the operational monitoring manager.

In certain embodiments, a high capability device, such as a gas monitoring device having a cellular and/or satellite communication option, may be utilized to bridge network portions within the gas monitoring system, for example to support gas monitoring devices and/or gas stations in an area of the facility where a full data manager is not desired, or not available. The extension of the network using a high capability bridging device allows for operations of the network to be supported beyond the original installation limits, for example to respond to temporary and/or highly transient changes in the gas monitoring environment for the facility, without waiting for or requiring a full installation using an anchor or data manager.

The methods and systems described herein may be deployed in part or in whole through a machine having a computer, computing device, processor, circuit, and/or server that executes computer readable instructions, program codes, instructions, and/or includes hardware configured to functionally execute one or more operations of the methods and systems disclosed herein. The terms computer, computing device, processor, circuit, and/or server, as utilized herein, should be understood broadly.

Any one or more of the terms computer, computing device, processor, circuit, and/or server include a computer of any type, capable to access instructions stored in communication thereto such as upon a non-transient computer readable medium, whereupon the computer performs operations of systems or methods described herein upon executing the instructions. In certain embodiments, such instructions themselves comprise a computer, computing device, processor, circuit, and/or server. Additionally or alternatively, a computer, computing device, processor, circuit, and/or server may be a separate hardware device, one or more computing resources distributed across hardware devices, and/or may include such aspects as logical circuits, embedded circuits, sensors, actuators, input and/or output devices, network and/or communication resources, memory resources of any type, processing resources of any type, and/or hardware devices configured to be responsive to determined conditions to functionally execute one or more operations of systems and methods herein.

Network and/or communication resources include, without limitation, local area network, wide area network, wireless, internet, or any other known communication resources and protocols. Example and non-limiting hardware, computers, computing devices, processors, circuits, and/or servers include, without limitation, a general purpose computer, a server, an embedded computer, a mobile device, a virtual machine, and/or an emulated version of one or more of these. Example and non-limiting hardware, computers, computing devices, processors, circuits, and/or servers may be physical, logical, or virtual. A computer, computing device, processor, circuit, and/or server may be: a distributed resource included as an aspect of several devices; and/or included as an interoperable set of resources to perform described functions of the computer, computing device, processor, circuit, and/or server, such that the distributed resources function together to perform the operations of the computer, computing device, processor, circuit, and/or server. In certain embodiments, each computer, computing device, processor, circuit, and/or server may be on separate hardware, and/or one or more hardware devices may include aspects of more than one computer, computing device, processor, circuit, and/or server, for example as separately executable instructions stored on the hardware device, and/or as logically partitioned aspects of a set of executable instructions, with some aspects of the hardware device comprising a part of a first computer, computing device, processor, circuit, and/or server, and some aspects of the hardware device comprising a part of a second computer, computing device, processor, circuit, and/or server.

A computer, computing device, processor, circuit, and/or server may be part of a server, client, network infrastructure, mobile computing platform, stationary computing platform, or other computing platform. A processor may be any kind of computational or processing device capable of executing program instructions, codes, binary instructions and the like. The processor may be or include a signal processor, digital processor, embedded processor, microprocessor or any variant such as a co-processor (math co-processor, graphic co-processor, communication co-processor and the like) and the like that may directly or indirectly facilitate execution of program code or program instructions stored thereon. In addition, the processor may enable execution of multiple programs, threads, and codes. The threads may be executed simultaneously to enhance the performance of the processor and to facilitate simultaneous operations of the application. By way of implementation, methods, program codes, program instructions and the like described herein may be implemented in one or more threads. The thread may spawn other threads that may have assigned priorities associated with them; the processor may execute these threads based on priority or any other order based on instructions provided in the program code. The processor may include memory that stores methods, codes, instructions and programs as described herein and elsewhere. The processor may access a storage medium through an interface that may store methods, codes, and instructions as described herein and elsewhere. The storage medium associated with the processor for storing methods, programs, codes, program instructions or other type of instructions capable of being executed by the computing or processing device may include but may not be limited to one or more of a CD-ROM, DVD, memory, hard disk, flash drive, RAM, ROM, cache and the like.

A processor may include one or more cores that may enhance speed and performance of a multiprocessor. In embodiments, the process may be a dual core processor, quad core processors, other chip-level multiprocessor and the like that combine two or more independent cores (called a die).

The methods and systems described herein may be deployed in part or in whole through a machine that executes computer readable instructions on a server, client, firewall, gateway, hub, router, or other such computer and/or networking hardware. The computer readable instructions may be associated with a server that may include a file server, print server, domain server, internet server, intranet server and other variants such as secondary server, host server, distributed server and the like. The server may include one or more of memories, processors, computer readable transitory and/or non-transitory media, storage media, ports (physical and virtual), communication devices, and interfaces capable of accessing other servers, clients, machines, and devices through a wired or a wireless medium, and the like. The methods, programs, or codes as described herein and elsewhere may be executed by the server. In addition, other devices required for execution of methods as described in this application may be considered as a part of the infrastructure associated with the server.

The server may provide an interface to other devices including, without limitation, clients, other servers, printers, database servers, print servers, file servers, communication servers, distributed servers, and the like. Additionally, this coupling and/or connection may facilitate remote execution of instructions across the network. The networking of some or all of these devices may facilitate parallel processing of program code, instructions, and/or programs at one or more locations without deviating from the scope of the disclosure. In addition, all the devices attached to the server through an interface may include at least one storage medium capable of storing methods, program code, instructions, and/or programs. A central repository may provide program instructions to be executed on different devices. In this implementation, the remote repository may act as a storage medium for methods, program code, instructions, and/or programs.

The methods, program code, instructions, and/or programs may be associated with a client that may include a file client, print client, domain client, internet client, intranet client and other variants such as secondary client, host client, distributed client and the like. The client may include one or more of memories, processors, computer readable transitory and/or non-transitory media, storage media, ports (physical and virtual), communication devices, and interfaces capable of accessing other clients, servers, machines, and devices through a wired or a wireless medium, and the like. The methods, program code, instructions, and/or programs as described herein and elsewhere may be executed by the client. In addition, other devices utilized for execution of methods as described in this application may be considered as a part of the infrastructure associated with the client.

The client may provide an interface to other devices including, without limitation, servers, other clients, printers, database servers, print servers, file servers, communication servers, distributed servers, and the like. Additionally, this coupling and/or connection may facilitate remote execution of methods, program code, instructions, and/or programs across the network. The networking of some or all of these devices may facilitate parallel processing of methods, program code, instructions, and/or programs at one or more locations without deviating from the scope of the disclosure. In addition, all the devices attached to the client through an interface may include at least one storage medium capable of storing methods, program code, instructions, and/or programs. A central repository may provide program instructions to be executed on different devices. In this implementation, the remote repository may act as a storage medium for methods, program code, instructions, and/or programs.

The methods and systems described herein may be deployed in part or in whole through network infrastructures. The network infrastructure may include elements such as computing devices, servers, routers, hubs, firewalls, clients, personal computers, communication devices, routing devices and other active and passive devices, modules, and/or components as known in the art. The computing and/or non-computing device(s) associated with the network infrastructure may include, apart from other components, a storage medium such as flash memory, buffer, stack, RAM, ROM and the like. The methods, program code, instructions, and/or programs described herein and elsewhere may be executed by one or more of the network infrastructural elements.

The methods, program code, instructions, and/or programs described herein and elsewhere may be implemented on a cellular network having multiple cells. The cellular network may either be frequency division multiple access (FDMA) network or code division multiple access (CDMA) network. The cellular network may include mobile devices, cell sites, base stations, repeaters, antennas, towers, and the like.

The methods, program code, instructions, and/or programs described herein and elsewhere may be implemented on or through mobile devices. The mobile devices may include navigation devices, cell phones, mobile phones, mobile personal digital assistants, laptops, palmtops, netbooks, pagers, electronic books readers, music players, and the like. These mobile devices may include, apart from other components, a storage medium such as a flash memory, buffer, RAM, ROM and one or more computing devices. The computing devices associated with mobile devices may be enabled to execute methods, program code, instructions, and/or programs stored thereon. Alternatively, the mobile devices may be configured to execute instructions in collaboration with other devices. The mobile devices may communicate with base stations interfaced with servers and configured to execute methods, program code, instructions, and/or programs. The mobile devices may communicate on a peer to peer network, mesh network, or other communications network. The methods, program code, instructions, and/or programs may be stored on the storage medium associated with the server and executed by a computing device embedded within the server. The base station may include a computing device and a storage medium. The storage device may store methods, program code, instructions, and/or programs executed by the computing devices associated with the base station.

The methods, program code, instructions, and/or programs may be stored and/or accessed on machine readable transitory and/or non-transitory media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory; optical storage such as CD, DVD; removable media such as flash memory (e.g., USB sticks or keys), floppy disks, magnetic tape, paper tape, punch cards, standalone RAM disks, Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, and the like.

Certain operations described herein include interpreting, receiving, and/or determining one or more values, parameters, inputs, data, or other information. Operations including interpreting, receiving, and/or determining any value parameter, input, data, and/or other information include, without limitation: receiving data via a user input; receiving data over a network of any type; reading a data value from a memory location in communication with the receiving device; utilizing a default value as a received data value; estimating, calculating, or deriving a data value based on other information available to the receiving device; and/or updating any of these in response to a later received data value. In certain embodiments, a data value may be received by a first operation, and later updated by a second operation, as part of the receiving a data value. For example, when communications are down, intermittent, or interrupted, a first operation to interpret, receive, and/or determine a data value may be performed, and when communications are restored an updated operation to interpret, receive, and/or determine the data value may be performed.

Certain logical groupings of operations herein, for example methods or procedures of the current disclosure, are provided to illustrate aspects of the present disclosure. Operations described herein are schematically described and/or depicted, and operations may be combined, divided, re-ordered, added, or removed in a manner consistent with the disclosure herein. It is understood that the context of an operational description may require an ordering for one or more operations, and/or an order for one or more operations may be explicitly disclosed, but the order of operations should be understood broadly, where any equivalent grouping of operations to provide an equivalent outcome of operations is specifically contemplated herein. For example, if a value is used in one operational step, the determining of the value may be required before that operational step in certain contexts (e.g. where the time delay of data for an operation to achieve a certain effect is important), but may not be required before that operation step in other contexts (e.g. where usage of the value from a previous execution cycle of the operations would be sufficient for those purposes). Accordingly, in certain embodiments an order of operations and grouping of operations as described is explicitly contemplated herein, and in certain embodiments re-ordering, subdivision, and/or different grouping of operations is explicitly contemplated herein.

The methods and systems described herein may transform physical and/or or intangible items from one state to another. The methods and systems described herein may also transform data representing physical and/or intangible items from one state to another.

The elements described and depicted herein, including in flow charts, block diagrams, and/or operational descriptions, depict and/or describe specific example arrangements of elements for purposes of illustration. However, the depicted and/or described elements, the functions thereof, and/or arrangements of these, may be implemented on machines, such as through computer executable transitory and/or non-transitory media having a processor capable of executing program instructions stored thereon, and/or as logical circuits or hardware arrangements. Example arrangements of programming instructions include at least: monolithic structure of instructions; standalone modules of instructions for elements or portions thereof; and/or as modules of instructions that employ external routines, code, services, and so forth; and/or any combination of these, and all such implementations are contemplated to be within the scope of embodiments of the present disclosure Examples of such machines include, without limitation, personal digital assistants, laptops, personal computers, mobile phones, other handheld computing devices, medical equipment, wired or wireless communication devices, transducers, chips, calculators, satellites, tablet PCs, electronic books, gadgets, electronic devices, devices having artificial intelligence, computing devices, networking equipment, servers, routers and the like. Furthermore, the elements described and/or depicted herein, and/or any other logical components, may be implemented on a machine capable of executing program instructions. Thus, while the foregoing flow charts, block diagrams, and/or operational descriptions set forth functional aspects of the disclosed systems, any arrangement of program instructions implementing these functional aspects are contemplated herein. Similarly, it will be appreciated that the various steps identified and described above may be varied, and that the order of steps may be adapted to particular applications of the techniques disclosed herein. Additionally, any steps or operations may be divided and/or combined in any manner providing similar functionality to the described operations. All such variations and modifications are contemplated in the present disclosure. The methods and/or processes described above, and steps thereof, may be implemented in hardware, program code, instructions, and/or programs or any combination of hardware and methods, program code, instructions, and/or programs suitable for a particular application. Example hardware includes a dedicated computing device or specific computing device, a particular aspect or component of a specific computing device, and/or an arrangement of hardware components and/or logical circuits to perform one or more of the operations of a method and/or system. The processes may be implemented in one or more microprocessors, microcontrollers, embedded microcontrollers, programmable digital signal processors or other programmable device, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a machine readable medium.

The computer executable code may be created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and computer readable instructions, or any other machine capable of executing program instructions.

Thus, in one aspect, each method described above and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or computer-readable instructions described above. All such permutations and combinations are contemplated in embodiments of the present disclosure.

While the disclosure has been disclosed in connection with the preferred embodiments shown and described in detail, various modifications and improvements thereon will become readily apparent to those skilled in the art. Accordingly, the spirit and scope of the present disclosure is not to be limited by the foregoing examples, but is to be understood in the broadest sense allowable by law.

1-63. (canceled)

64. A system, comprising:
a cooperative monitoring group of gas monitors, the cooperative monitoring group comprising:
a network of end points, the end points comprising at least one end point from each class selected from:
a gas monitor class, each gas monitor comprising a gas sensing element and a wireless communication interface;
a gas station class, each gas station comprising at least one gas monitor physical interface, and a wireless communication interface; and
a data manager class, each data manager comprising a wireless communication interface and an external communication interface; and
wherein the cooperative monitoring group is configured to operate as a mesh network, and wherein external communications of the cooperative monitoring group from end points of the gas station class and the gas monitor class are routed through end points of the data manager class; and
a fence-line comprising a plurality of gas monitors at selected locations.

65. The system of claim 64, wherein the selected location for at least one of the plurality of gas monitors comprises a fixed location.

66. The system of claim 64, wherein the selected location for at least one of the plurality of gas monitors comprises a mobile location.

67. The system of claim 66, wherein the selected location is determined in response to at least one signal strength value.

68. The system of claim 64, wherein each one of the plurality of gas monitors is configured to operate in at least one of a monitoring mode or an anchor mode.

69. The system of claim 68, further comprising a fence-line manager configured to determine operating modes for the plurality of gas monitors in response to a battery power value for at least one of the plurality of gas monitors.

70. The system of claim 64, further comprising a fence-line manager configured to monitor the plurality of gas monitors as a fence-line group.

71. The system of claim 64, further comprising a fence-line manager configured to determine a geofenced region in response to the selected locations.

72. The system of claim 64, further comprising a fence-line manager configured to monitor a geofenced region in response to the selected locations.
